(19) United States
(12) Patent Application Publication
(10) Pub. No.: US 2009/0247772 A1
(43) Pub. Date: Oct. 1, 2009

(54) Method for the Preparation of Escitalopram

(75) Inventors: Antonio Paulon, Venezia (IT); Ottorino De Lucchi, Padova (IT); Andrea Castellin, Mestrino (Padova) (IT); Fabrizio Fabris, Treviso (IT); Federico Sbrogio, Montecchio Maggiore (IT); Emanuele Ceron, Murelle di Villanova di Camposampiero (IT); Hans Petersen, Vanlose (DK); Robert Dancer, Hvidovre (DK)

Correspondence Address: DARBY & DARBY P.C., P.O. BOX 770, Church Street Station, New York, NY, 10008-0770, US

(73) Assignee: H. Lundbeck A/S, Valby-Copenhagen (DK)

(21) Appl. No.: 12/085,063

(22) PCT Filed: Nov. 14, 2006
(86) PCT No.: PCT/DK2006/050067

§ 371 Date: Oct. 29, 2008

(30) Foreign Application Priority Data
Nov. 14, 2005 (DK) PA 200501581

(51) Int. Cl. C07D 307/93; C07D 307/87

(52) U.S. Cl. 549/459; 549/469

(57) ABSTRACT

The invention relates to intermediates and the use thereof in a method for the preparation of escitalopram:

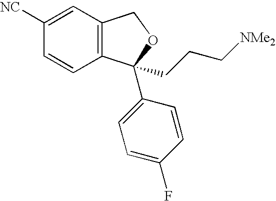

The present invention relates to novel intermediates and the use thereof in a novel method for the preparation of escitalopram.

### BACKGROUND OF THE INVENTION

Citalopram is a well-known antidepressant drug that has now been on the market for some years.

It is a selective, centrally acting serotonin (5-hydroxytryptamine; 5-HT) reuptake inhibitor, accordingly having antidepressant activities.

Citalopram was first disclosed in DE 2,657,013, corresponding to U.S. Pat. No. 4,136,193. This patent publication i.a. outlines a process for preparation of citalopram from the corresponding 5-bromo-derivative by reaction with cuprous cyanide in a suitable solvent and by alkylation of 5-bromo-phtalane.

U.S. Pat. No. 4,943,590 corresponding to EP-B1-347 066 describes two processes for the preparation of escitalopram (S-enantiomer of citalopram). Both processes use the racemic diol having the formula

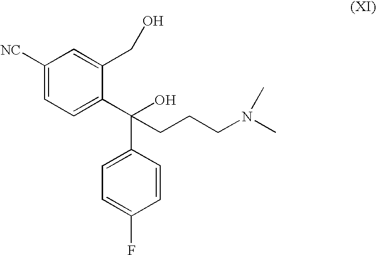

as starting material. According to the first process, the diol of formula (XI) is reacted with an enantiomerically pure acid derivative, such as (+) or (−)-α-methoxy-α-trifluoromethyl-phenylacetyl chloride to form a mixture of diastereomeric esters, which are separated by HPLC or fractional crystallization, whereupon the ester with the correct stereochemistry is enantioselectively converted into escitalopram. According to the second process, the diol of formula (XI) is separated into the enantiomers by stereoselective crystallization with an enantiomerically pure acid such as (+)-di-p-toluoyltartaric acid, whereupon the S-enantiomer of the diol of the formula (XI) is enantioselectively converted to escitalopram.

Escitalopram, a compound of formula VIII

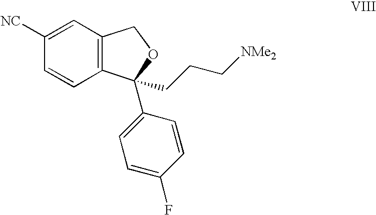

has now been developed as an antidepressant. Hence, there is a desire for an improved method for preparation of escitalopram.

A new method for the preparation of escitalopram has now been found which has the following advantages: the reaction steps are suitable on a large scale, a high yield can be obtained and the starting materials are widely available.

### SUMMARY OF THE INVENTION

The objective of the present invention is to provide a new and commercially interesting method for the preparation of escitalopram.

Accordingly, one object of the present invention relates to a method for the preparation of a compound of formula VI

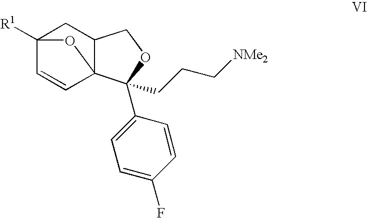

wherein R¹ is selected from functionalities that can be transformed into a nitrile group by conventional methods,
comprising allowing a compound of formula V

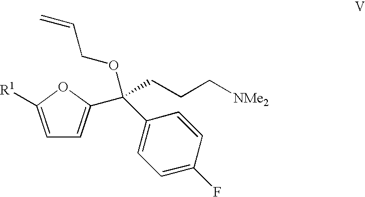

wherein R¹ is as defined above, to react to produce said compound of formula VI, optionally by heating, optionally in the presence of a Lewis acid and optionally in a suitable solvent.

Another object of the present invention relates to a method for the manufacturing of escitalopram.

Another object of the present invention relates to a compound of formula VI

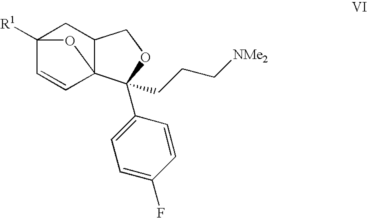

wherein R¹ is as defined above.

Another object of the present invention relates to a compound of formula V

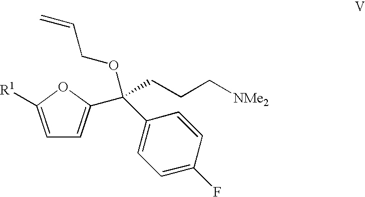

wherein R¹ is as defined above.

Another object of the present invention relates to a compound of formula IV

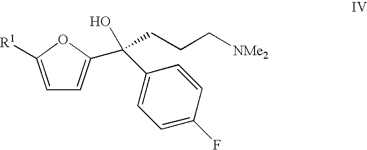

wherein R¹ is as defined above.

Another object of the present invention relates to a compound of formula III

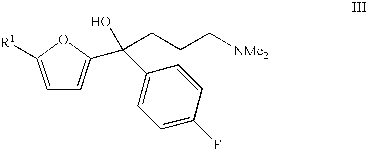

wherein R¹ is as defined above.

Another object of the present invention relates to a compound of formula II

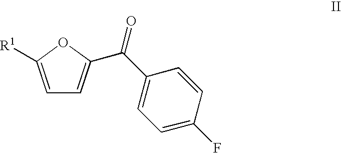

wherein R¹ is as defined above.

Another object of the present invention relates to a compound of formula I

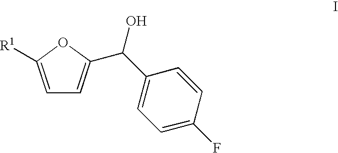

wherein R¹ is as defined above.

Another object of the present invention relates to the use of one or more compounds of formula I, II, III, IV, V or VI in a method for the preparation of escitalopram.

Another object of the present invention relates to a pharmaceutical composition comprising escitalopram produced by a process comprising one or more of the methods according to the present invention.

### DETAILED DESCRIPTION OF THE INVENTION

According to a particular embodiment of the present invention the compound of formula VI is a compound of formula VIa or VIb

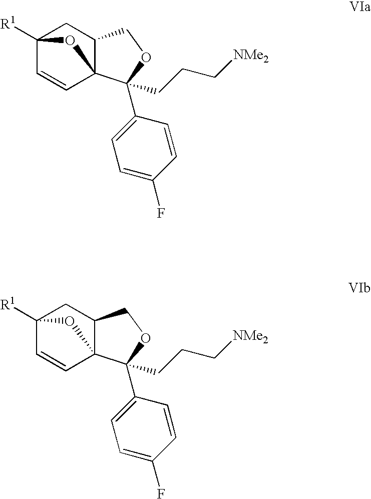

or any mixture of VIa and VIb, wherein R¹ is as defined above.

According to another particular embodiment of the present invention R¹ is selected from functionalities that can be transformed into a nitrile group by conventional methods, such as carboxylic acid derivatives, preferably esters (—COOR², wherein R² is selected from C₁₋₆-alkyl, optionally substituted aryl or optionally substituted heteroaryl), amides, preferably (—COONHR³, wherein R³ is selected from hydrogen and C₁₋₆-alkyl), oxazolines, carbaldehyde derivatives, preferably (—CHO) or derivatives thereof, preferably dioxolans, acetals or aminals, and halogens, preferably Cl, Br, or I.

According to still another particular embodiment of the present invention R¹ is 1,3-dioxolan-2-yl.

According to a particular embodiment of the present invention the Lewis acid in the method for the preparation of a compound of formula VI as described above, is selected from BF₃.Et₂O or anhydrous ZnCl₂, TiCl₄, AlCl₃, SnCl₄ or the likes.

According to a particular embodiment of the present invention the solvent in the method for the preparation of a compound of formula VI as described above, is selected from CH₂Cl₂, CHCl₃, toluene or the likes.

According to a particular embodiment of the present invention the compound of formula V is prepared by reacting a compound of formula IV

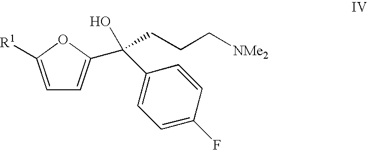

wherein R¹ is as defined above, with an allylating agent.

According to another particular embodiment of the present invention the allylating agent in the method for the preparation of a compound of formula V as described above, is selected from allyl bromide or allyl chloride.

According to a particular embodiment of the present invention the compound of formula IV is prepared by resolution of a compound of formula III

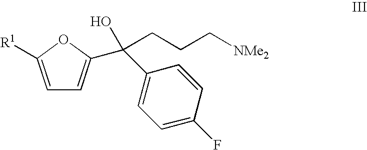

wherein R¹ is as defined above.

According to another particular embodiment of the present invention the resolution in the method for the preparation of a compound of formula IV as described above, is selected from classic resolution, enzymatic resolution or chiral chromatography, such as simulated moving bed resolution.

According to a particular embodiment of the present invention the compound of formula III is prepared by reacting a compound of formula II

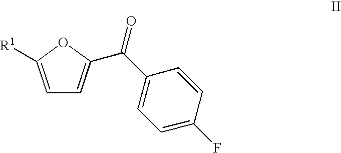

wherein R¹ is as defined above, with dimethylaminopropyl magnesium chloride.

According to a particular embodiment of the present invention the compound of formula II is prepared by reacting a compound of formula I

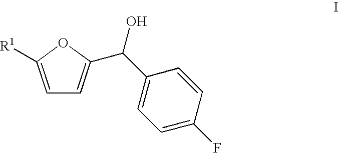

wherein R¹ is as defined above, with an oxidising agent in a suitable solvent.

According to another particular embodiment of the present invention the oxidising agent in the method for the preparation of a compound of formula II as described above, is manganese dioxide.

According to still another particular embodiment of the present invention the solvent in the method for the preparation of a compound of formula II as described above, is dichloromethane.

According to a particular embodiment of the present invention the compound of formula I is prepared by reacting a compound of formula IX

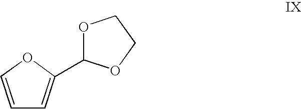

and a compound of formula X

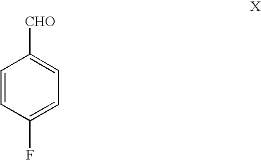

in the presence of a strong base in a suitable solvent.

According to another particular embodiment of the present invention the strong base in the method for the preparation of a compound of formula I as described above, is an organometallic agent.

According to another particular embodiment of the present invention the strong base in the method for the preparation of a compound of formula I as described above, is selected from LDA, LHMDS, methyl lithium, butyl lithium, n-butyl lithium, n-hexyl lithium or cyclohexyl lithium.

According to still another particular embodiment of the present invention the solvent in the method for the preparation of a compound of formula I as described above, is THF.

According to a particular embodiment of the present invention the compound of formula VI is reacted under acidic conditions to produce a compound of formula VII

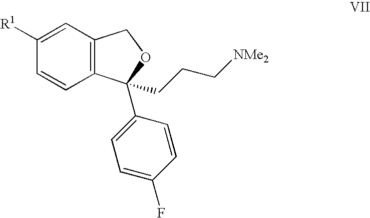

wherein R¹ is defined above.

According to another particular embodiment of the present invention the acidic conditions in the method for the preparation of a compound of formula VII as described above, are generated by an acid selected from Lewis acids, organic acids or mineral acids or a mixture thereof.

According to a particular embodiment of the present invention R¹ of the compound of formula VII is transformed into a nitrile group to produce escitalopram, a compound of formula VIII

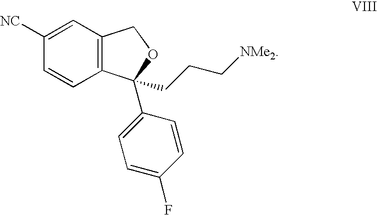

According to a particular embodiment of the present invention the compound of formula VIII is optionally further purified and optionally converted to a pharmaceutically acceptable form.

According to a particular embodiment of the present invention a compound of formula VI is S-{3-[7-[1,3]dioxolan-2-yl-2-(4-fluoro-phenyl)-3,10-dioxa-tricyclo[5.2.1.0^{1,5}]dec-8-en-2-yl]-propyl}-dimethyl-amine.

According to a particular embodiment of the present invention a compound of formula V is S-[4-allyloxy-4-(5-[1,3]dioxolan-2-yl-furan-2-yl)-4-(4-fluoro-phenyl)-butyl]-dimethyl-amine.

According to a particular embodiment of the present invention a compound of formula IV is S-4-dimethylamino-1-(5-[1,3]dioxolan-2-yl-furan-2-yl)-1-(4-fluoro-phenyl)-butan-1-ol.

According to a particular embodiment of the present invention a compound of formula III is 4-dimethylamino-1-(5-[1,3]dioxolan-2-yl-furan-2-yl)-1-(4-fluoro-phenyl)-butan-1-ol.

According to a particular embodiment of the present invention a compound of formula II is (5-[1,3]dioxolan-2-yl-furan-2-yl)-(4-fluoro-phenyl)-methanone.

According to a particular embodiment of the present invention a compound of formula I is (5-[1,3]dioxolan-2-yl-furan-2-yl)-(4-fluoro-phenyl)-methanol.

According to a particular embodiment of the present invention escitalopram is prepared by a method comprising one or more of the steps a) to i)

a)
reacting a compound of formula IX

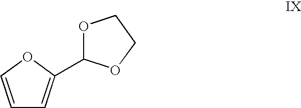

and a compound of formula X

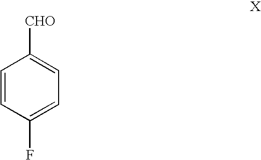

in the presence of a strong base in a suitable solvent, to produce a compound of formula I wherein R¹ is as described above;
b)
reacting a compound of formula I

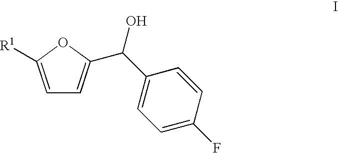

wherein R¹ is as defined above, with an oxidising agent in a suitable solvent, to produce a compound of formula II

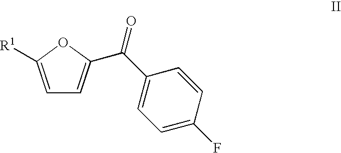

wherein R¹ is as described above;
c)
reacting a compound of formula II

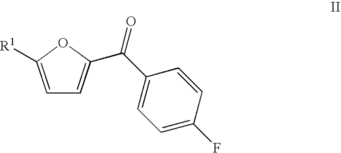

wherein R¹ is as defined above, with dimethylaminopropyl magnesium chloride, to produce a compound of formula III

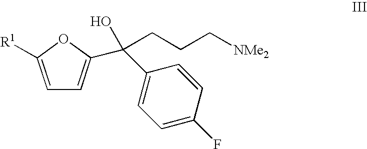

wherein R¹ is as described above;
d)
resolution of a compound of formula III

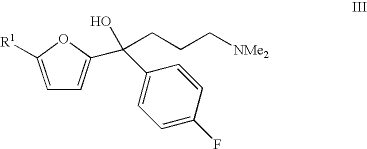

wherein R¹ is as defined above, to produce a compound of formula IV

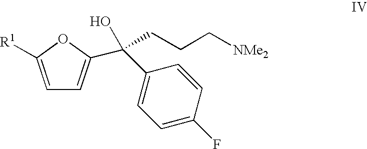

wherein R¹ is as described above;
e)
reacting a compound of formula IV

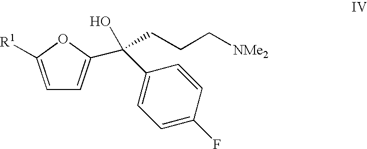

wherein R¹ is as defined above, with an allylating agent, to produce a compound of formula V;

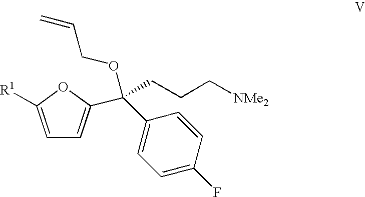

f)
allowing a compound of formula V

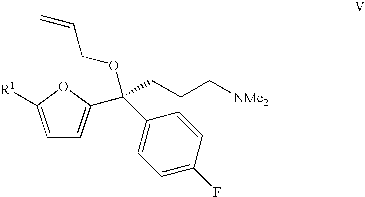

wherein R¹ is as defined above, to react to produce a compound of formula VI

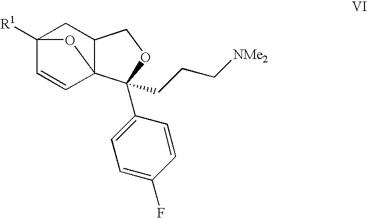

wherein R¹ is as described above, optionally by heating, optionally in the presence of a Lewis acid and optionally in a suitable solvent;
g)
reacting a compound of formula VI

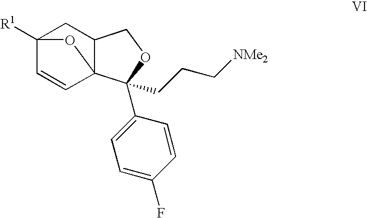

under acidic conditions to produce a compound of formula VII

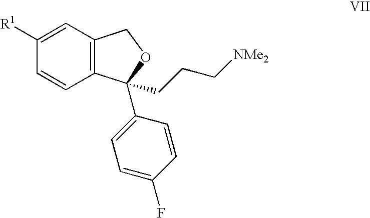

wherein R¹ is defined above;
h)
transforming R¹ of a compound of formula VII
into a nitrile group to produce escitalopram, a compound of formula VIII

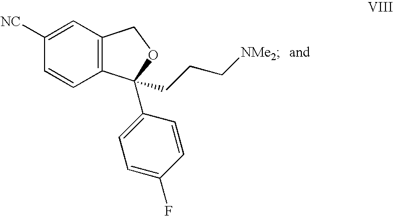

i)
optionally further purifying and/or optionally converting the compound of formula VIII to a pharmaceutically acceptable form.

The term “heating” as used in the present invention designates any method, preferably conventional methods such as conventional heating, microwave or ultrasound that can raise the temperature of the reaction mixture.

The term “allylating agent” in the method for the preparation of a compound of formula V designates a source of allyl cation or a equivalent thereof, such as allyl bromide and allyl chloride.

The term “resolution” in the method for the preparation of a compound of formula IV designates methods, such as classic resolution, enzymatic resolution or chiral chromatography, such as simulated moving bed resolution.

The term “strong base” in the method for the preparation of a compound of formula I designates a base capable of deprotonating the α-position of a furan, such as LHMDS or butyl lithium.

The term “oxidising agent” in the method for the preparation of a compound of formula II designates a reagent capable of oxidising a secondary alcohol to the corresponding ketone, such as manganese dioxide.

The term “C₁₋₆-alkyl” designates a branched or unbranched alkyl group having from one to six carbon atoms, including but not limited to methyl, ethyl, prop-1-yl, prop-2-yl, 2-methyl-prop-1-yl, 2-methyl-prop-2-yl, 2,2-dimethyl-prop-1-yl, but-1-yl, but-2-yl, 3-methyl-but-1-yl, 3-methyl-but-2-yl, pent-1-yl, pent-2-yl, pent-3-yl, hex-1-yl, hex-2-yl and hex-3-yl

The term “optionally substituted aryl” designates monocyclic or bicyclic aromatic systems of 5-10 carbon atoms, including but not limited to phenyl and naphthyl, which may be optionally substituted, such as with 0, 1, 2, 3 or 4 substituents independently selected from the group consisting of amino, halogen, cyano or C₁₋₆-alkyl.

The term “optionally substituted heteroaryl” designates monocyclic or bicyclic heteroaromatic systems of 5-10 atoms selected from 1, 2, 3, 4, 5, 6, 7, 8 or 9 carbon atoms and 1, 2, 3 or 4 heteroatoms independently selected from N, S, or O, including but not limited to pyridine, pyrrole, pyrimidine, quinoline, indole, thiophene, furan, imidazoles such as 3H-imidazol and 1H-imidazol, triazoles such as [1,2,3]triazole and [1,2,4]triazole, tetrazoles such as 2H-tetrazole and oxazole, which may be optionally substituted, such as with 0, 1, 2, 3 or 4 substituents independently selected from the group consisting of halogen, cyano, amino or C₁₋₆-alkyl.

The term “pharmaceutically acceptable form” of the compound of formula VIII designates any form of said compound that can be formulated into a pharmaceutical composition, such as a pharmaceutically acceptable salt thereof, such as oxalate, HBr or HCl, or as the free base.

R¹ may be transformed into a nitrile group according to any method known to the person skilled in the art.

When R¹ is halogen, in particular bromo or chloro, transformation to a nitrile may be carried out as described in U.S. Pat. No. 4,136,193, WO 00/13648, WO 00/11926 and WO 01/02383.

According to U.S. Pat. No. 4,136,193 transformation of a bromo group to a nitrile group, is carried out by reaction with CuCN.

When R¹ is a carbaldehyde derivative, in particular —CHO, transformation to a nitrile may be carried out as described in WO 99/30548.

Classic resolution may be performed as described in U.S. Pat. No. 4,943,590 corresponding to EP-B1-347 066.

Chiral chromatography may be performed as described in WO03006449.

Enzymatic resolution may be performed as described in WO2004014821.

### EXAMPLES

¹H NMR and ¹³C NMR spectra were recorded using Bruker AV300 spectrometer operating at 300 and 75 MHz respectively and a Bruker AV500 spectrometer operating at 500 MHz and 125 MHz respectively. The multiplicities are indicated as: s (singlet), bs (broad singlet), d (doublet), dd (double doublet), t (triplet), etc. The frequencies of resonance are indicated in 6 ppm using TMS as reference (0 ppm).

The HPLC analyses were run on different systems.

For “HPLC (Lichrosorb RP8)” a system equipped with Lichrosorb RP8 column (5×250 mm) was used. The eluant was a 50:50 mixture of H₂O:CH₃CN buffered at pH=3 (Triethylammonium phosphate) and the flow rate was 1.00 mL/min.

For “HPLC (Chiralcel OD)” a system equipped with Chiralcel OD column (5×250 mm) was used. Packing composition: cellulose tris(3,5-dimethylphenylcarbamate) coated on 10 μm silica-gel. The eluant was a mixture of the following: Heptane (98.4%), ethanol (1.5%), diethylamine (0.1%). The flow rate was 1.00 mL/min.

For “HPLC (Chirpak AD)” a system equipped with Chiralpak AD column (5×250 mm) was used. Packing composition: Amylose tris(3,5-dimethylphenylcarbamate) coated on 10 μm silica-gel. The eluant was a mixture of the following: Heptane (90%), ethanol (10%), diethylamine (0.1%). The flow rate was 1.00 mL/min.

All chemicals were purchased from Aldrich or Fluka and used without purification. Where specified the chemicals were purified using the methods described in Perrin, D. D.; Armarego, W. L. F. “Purification of laboratory chemicals”, Pergamon Press, Oxford, 1988.

### Example 1

### (5-[1,3]Dioxolan-2-yl-furan-2-yl)-(4-fluoro-phenyl)-methanol (1)

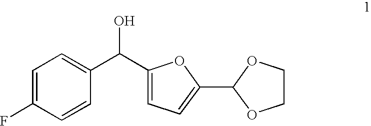

In a 1 L two-neck round bottomed flask, equipped with magnetic bar, dried and in argon atmosphere, n-butyllithium (2.5 M solution in hexane, 144 mL, 0.36 mol, 1 eq) was added over 40 minutes to a well-stirred solution of dry diisopropylamine (47.1 mL, 0.36 mol, 1 eq) in dry tetrahydrofuran (THF) (300 mL) at −20° C. The mixture was stirred for 20 minutes, cooled to −78° C. and a solution of 2-furan-2-yl-[1,3]dioxolane (50 g, 0.36 mol, 1 eq.) in dry THF (100 mL) was added dropwise. During the whole operation the reaction temperature was maintained at −78° C., and the stirring was continued for 30 minutes. A solution of 4-fluoro-benzaldehyde (45.5 g, 0.36 mol, 1 eq) in dry THF (100 mL) was then added dropwise at this temperature and the stirring was continued for 1 hour. The temperature was allowed to rise to room temperature over 16 hours. The mixture was concentrated under reduced pressure, dissolved into ether (600 mL), washed with water (3×300 mL) and brine (2×200 mL). The organic layers were collected, dried (MgSO₄), filtered and concentrated affording a yellow oil. Crystallisation from ether/n-hexane afforded (1) (92 g, 97%) as a white solid.

¹H (300 MHz, CDCl₃) (δ ppm): 7.42 (dd, J_{HH}=5.3 Hz, J_{HF}=8.8 Hz, 2H), 7.07 (t, J_{HH}=8.8 Hz, J_{HF}=8.8 Hz, 2H), 6.37 (d, J_{HH}=3.2 Hz, 1H), 6.02 (d, J_{HH}=3.2 Hz, 1H), 5.89 (s, 1H), 5.79 (d, J_{HH}=4.0 Hz, 1H), 4.12-3.96 (m, 4H), 2.71 (d, J_{HH}=4.0 Hz, 1H).

¹³C (75 MHz, CDCl₃) (δ ppm): 162 (d, ¹J_{CF}=246 Hz), 157, 151, 136 (d, ⁴J_{CF}=3 Hz), 128 (d, ³J_{CF}=8 Hz), 115 (d, ²J_{CF}=²¹ Hz), 109, 108, 98, 69, 65.

HPLC (Lichrosorb RP8): r.t.=4.39 min.

### Example 2

### (5-[1,3]Dioxolan-2-yl-furan-2-yl)-(4-fluoro-phenyl)-methanone (2)

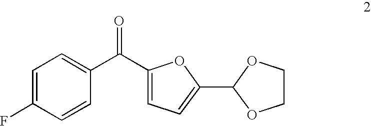

In a two-necked round-bottomed flask equipped with condenser and magnetic bar, a mixture of primary alcohol (1) (176 g, 0.7 mol) and manganese (IV) dioxide (148 g, 1.4 mol, 2 eq.) in dichloromethane (DCM) (300 mL) was heated at reflux overnight. The mixture was then cooled, filtered through a bed of celite and concentrated under reduced pressure to give a yellow oil. Crystallisation from methanol afforded ketone (2) as a white solid (182.1 g, 99%).

¹H (300 MHz, CDCl₃) (δ ppm): 8.00 (dd, J_{HH}=8.8 Hz, J_{HF}=5.5 Hz, 2H), 7.21 (d, J_{HH}=3.5 Hz, 1H), 7.18 (t, J_{HH}=8.8 Hz, J_{HF}=8.8 Hz, 2H), 6.63 (d, J_{HH}=3.5 Hz, 1H), 6.03 (s, 1H), 4.19-3.98 (m, 4H).

¹³C (75 MHz, CDCl₃) (δ ppm): 181, 166 (d, ¹J_{CF}=254 Hz), 156, 153, 133 (d, ⁴J_{CF}=3 Hz), 132 (d, ³J_{CF}=9 Hz), 121, 116 (d, ²J_{CF}=22 Hz), 111, 98, 66 (2C).

HPLC (Lichrosorb RP8): r.t.=5.96 min.

### Example 3

### 4-Dimethylamino-1-(5-[1,3]dioxolan-2-yl-furan-2-yl)-1-(4-fluoro-phenyl)-butan-1-ol (3)

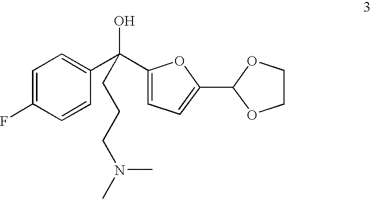

In a 1 L round bottomed flask equipped with magnetic bar and condenser, a mixture of 3-(dimethylamino)propyl-1-chloride hydrochloride (DMPC.HCl) (260 g, 1.65 mol) and aqueous NaOH solution (240 g, 30% w/v, 1.8 mol, 1.1 eq.) was heated at 45-50° C. for 2 hours. The mixture was then extracted with ether (3×400 mL) and the collected organic phase was dried (solid NaOH) and filtered. Distillation of ether at atmospheric pressure afforded DMPC as a colourless oil (160 g, 80%).

In a dry three neck round bottomed flask equipped with magnetic bar, thermometer, condenser and in argon atmosphere, a solution of DMPC (140 g, 1.14 mol, 3 eq) in dry THF (350 mL) was added dropwise over 1 hour to a mixture of magnesium turnings (27.36 g, 1.14 mol, 3 eq.) in dry THF (150 mL). The mixture was heated at reflux until the magnesium had been consumed, and then cooled to 0° C. using an ice bath. A solution of ketone 2 (100 g, 0.38 mol, 1 eq.) in dry THF (150 mL) was added over 2 hours and the temperature was allowed to rise to room temperature. After 16 hours saturated aqueous ammonia chloride solution (300 mL) was added and the mixture was extracted with ether (3×400 mL). The organic layers were collected, washed with water (3×400 mL), brine (2×400 mL), and then dried (MgSO₄), filtered and concentrated under reduced pressure to give a yellow oil. Crystallization from n-heptane afforded alcohol (3) (129.4 g, 98%) as a white solid.

¹H-NMR (300 MHz, CDCl₃) (δ ppm): 7.54 (dd, J_{HH}=8.6 Hz, J_{HF}=5.5 Hz, 2H), 7.00 (t, J_{HH}=8.6 Hz, J_{HF}=8.6 Hz, 2H), 6.34 (d, J_{HH}=3.3 Hz, 1H), 6.18 (d, J_{HH}=3.3 Hz, 1H), 5.92 (s, 1H), 4.13-3.95 (m, 4H), 2.57-2.45 (m, 1H), 2.36-2.20 (m, 3H), 2.17 (s, 6H), 1.58-1.46 (m, 2H).

¹³C (75 MHz, CDCl₃) (δ ppm): 162 (d, ¹J_{CF}=244 Hz), 161, 150, 142 (d, ⁴J_{CF}=3 Hz), 128 (d, ³J_{CF}=8 Hz), 115 (d, ²J_{CF}=21 Hz), 109, 107, 98, 74, 65, 60, 45, 42, 23.

HPLC (Lichrosorb RP8): r.t.=2.495 min.

HPLC (Chiralcel OD): r.t.=13.95 min. and r.t.=24.99 min.

### Example 4

### S-4-Dimethylamino-1-(5-[1,3]dioxolan-2-yl-furan-2-yl)-1-(4-fluoro-phenyl)-butan-1-ol (5)

### R-4-Dimethylamino-1-(5-[1,3]dioxolan-2-yl-furan-2-yl)-1-(4-fluoro-phenyl)-butan-1-ol (4)

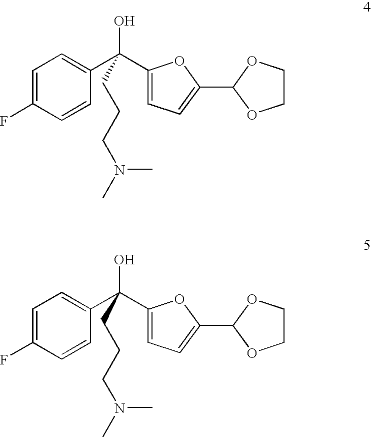

The separation of the racemic mixture to give the two enantiomers was performed using chiral chromatography (Simulated Moving Bed).

HPLC (Chiralcel OD): r.t.=14.03 min. (99.55%) for R-alcohol (4)

HPLC (Chiralcel OD): r.t.=25.89 min. (98.33%) for S-alcohol (5)

### Example 5

### S-[4-Allyloxy-4-(5-[1,3]dioxolan-2-yl-furan-2-yl)-4-(4-fluoro-phenyl)-butyl]-dimethyl-amine (6)

### R-[4-Allyloxy-4-(5-[1,3]dioxolan-2-yl-furan-2-yl)-4-(4-fluoro-phenyl)-butyl]-dimethyl-amine (9)

### [4-Allyloxy-4-(5-[1,3]dioxolan-2-yl-furan-2-yl)-4-(4-fluoro-phenyl)-butyl]-dimethyl-amine (6,9)

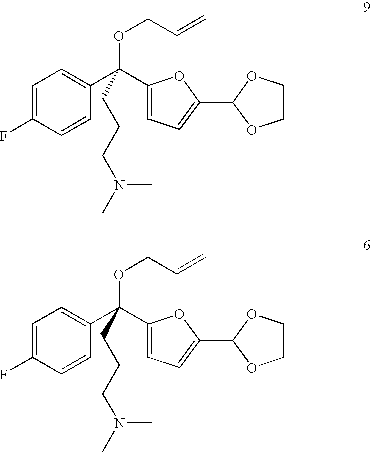

In a two necked round bottomed flask equipped with magnetic stirrer, condenser and under a nitrogen atmosphere, potassium hydride (3.60, g 31.5 mmol, 3 eq., c.a. 35% W/W dispersion in mineral oil) was washed three times with dry n-hexane, and then dry THF (20 mL) was added. A solution of S-alcohol (5) (3.63 g, 10.4 mmol) in dry THF (25 mL) was added dropwise and the resulting mixture was heated at reflux for 2 hours. The mixture was then cooled to room temperature. The stirring was stopped and the mixture was allowed to settle. The excess of potassium hydride was removed by decantation. The THF solution of alcoholate was transferred to a new well dry three necked round bottomed flask equipped with magnetic stirrer and condenser and 18-crown-6(1,4,7,10,13,16-hexaoxacyclootadecane) (2.77 g, 10.4 mmol, 1 eq.) was added and the mixture was heated at reflux for 20 minutes. The reaction was then cooled to room temperature and allyl bromide (1.09 mL, 12.47 mmol, 1.2 eq.) of was added portionwise (0.2 eq. every 10 minutes). The progress of the reaction was monitored by HPLC. The mixture was then diluted with ether (100 mL), washed with water (3×50 mL) and brine (2×30 mL). The organic layer was then dried (MgSO₄) and concentrated under reduced pressure affording the S-allyl derivative (6) (3.75 g, 93% by HPLC) as a red oil. The product was used in the next step without any further purification.

Using the same procedure, the R-derivative (9) and the racemic mixture of [4-allyloxy-4-(5-[1,3]dioxolan-2-yl-furan-2-yl)-4-(4-fluoro-phenyl)-butyl]-dimethyl-amine were synthesised from R-alcohol (4) (yield 91% by HPLC) and from racemic alcohol (3) (yield 92% by HPLC) respectively.

¹H (500 MHz, CDCl₃) (δ ppm): 7.33 (dd, J_{HH}=8.9 Hz, J_{HF}=5.4 Hz, 2H), 7.00 (t, J_{HH}=8.9 Hz, J_{HF}=8.9 Hz, 2H), 6.38 (d, J_{HH}=2.35 Hz, 1H), 6.29 (d, J_{HH}=2.35 Hz, 1H), 5.90-5.80 (m, 2H), 4.1-3.8 (AB SYSTEM, 2H), 4.1-3.8 (m, 4H), 1.41 (d, J_{HH}=2.83 Hz, 2H), 2.40-2.30 (m, 1H), 2.20-2.12 (m, 2H), 2.13-2.02 (m, 7H), 1.45-1.37 (m, 1H), 1.20-1.11 (m, 1H).

¹³C (125 MHz, CDCl₃) (δ ppm): 161 (d, ¹J_{CF}=255 Hz), 156, 150, 137 (d, ⁴J_{CF}=3 Hz), 134, 128 (d, 3J_{CF}=8 Hz), 115, 114 (d, ²J_{CF}=21 Hz), 109, 108, 97, 78, 64 (2C), 63, 59, 45, 34, 20.

HPLC (Lichrosorb RP8): r.t.=2.62 min.

### Example 6

### S-{3-[7-[1,3]Dioxolan-2-yl-2-(4-fluoro-phenyl)-3,10-dioxa-tricyclo[5.2.1.0^{1,5}]dec-8-en-2-yl]-propyl}-dimethyl-amine (7)

### R-{3-[7-[1,3]Dioxolan-2-yl-2-(4-fluoro-phenyl)-3,10-dioxa-tricyclo[5.2.1.0^{1,5}]dec-8-en-2-yl]-propyl}-dimethyl-amine (10)

### {3-[7-[1,3]Dioxolan-2-yl-2-(4-fluoro-phenyl)-3,10-dioxa-tricyclo[5.2.1.0^{1,5}]dec-8-en-2-yl]-propyl}-dimethyl-amine (7,10)

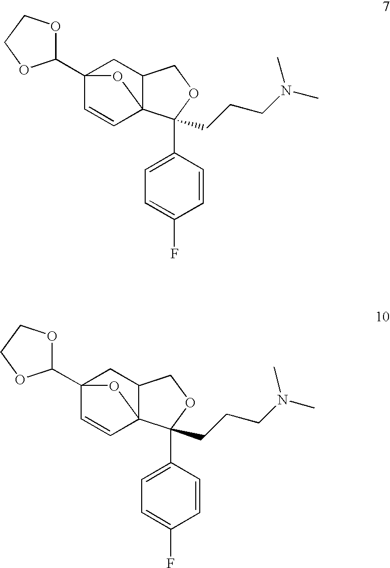

In a two-necked round-bottomed flask equipped with magnetic bar and condenser a solution of the S—O-allyl derivative (6) (3.75 g, 9.6 mmol) in toluene (15 mL) was heated at 85-95° C. overnight. The solution was concentrated under reduced pressure affording the two exo products of (7) as red oil (3.7 g, 99%).

The Diels Alder products were used in the next step without any further purification.

The toluene solution containing S-derivative (7) can also be used as is in the next step without any further treatment.

The Diels Alder reaction was also performed on the R-derivative (9) and on the racemic mixture giving (10) (yield 99%) and the racemic mixture (9), (10) (yield 99%).

¹H (500 MHz, CDCl₃) (δ ppm): 7.44 (dd, J_{HH}=8.9 Hz, J_{HF}=5.2 Hz, 2H), 7.28 (dd, J_{HH}=8.9 Hz, J_{HF}=5.2 Hz, 2H), 7.05 (t, J_{HH}=8.9 Hz, J_{HF}=8.9 Hz, 2H), 6.92 (t, J_{HH}=8.9 Hz, J_{HF}=8.9 Hz, 2H), 6.70 (d, J_{HH}=5.6 Hz, 1H), 6.46 (d, J_{HH}=5.6 Hz, 1H), 6.29 (d, J_{HH}=5.6 Hz, 1H), 5.96 (d, J_{HH}=5.6 Hz, 1H), 5.25 (s, 1H), 5.12 (s, 1H), 4.42 (t, J_{HH}=8.5 Hz, J_{HH}=8.5 Hz, 1H), 4.29 (t, J_{HH}=8.5 Hz, J_{HH}=8.5 Hz, 1H), 4.10-3.80 (m, 8H), 3.72 (dd, J_{HH}=8.5 Hz, J_{HH}=9.9 Hz, 1H), 3.62 (dd, J_{HH}=8.0 Hz, J_{HH}=9.9 Hz, 1H), 2.49 (dddd, J_{HH}=9.4 Hz, J_{HH}=8.0 Hz, J_{HH}=7.5 Hz, J_{HH}=3.3 Hz, 1H), 2.30-1.93 (m, 22H), 1.91-1.82 (m, 1H), 1.80-1.71 (m, 2H), 1.67-1.36 (m, 4H).

¹³C (125 MHz, CDCl₃) (δ ppm): 162 (d, ¹J_{CF}=245 Hz), 161 (d, ¹J_{CF}=244 Hz), 139 (d, ⁴J_{CF}=3 Hz), 137 (d, ⁴J_{CF}=3 Hz), 137, 136, 135, 134, 129 (d, ³J_{CF}=8 Hz, 2C), 128 (d, ³J_{CF}=8 Hz, 2C), 115 (d, ²J_{CF}=22 Hz, 2C), 114 (d, ²J_{CF}=22 Hz, 2C),103 (2C), 102 (2C), 92 (2C), 85, 84, 73, 72, 66 (4C), 60 (2C), 47 (1C), 46 (4C), 45, 39, 33, 32, 31, 23, 22.

### Example 7

### S-1-(3-Dimethylamino-propyl)-1-(4-fluoro-phenyl)-1,3-dihydro-isobenzofuran-5-carbaldehyde (8a)

### R-1-(3-Dimethylamino-propyl)-1-(4-fluoro-phenyl)-1,3-dihydro-isobenzofuran-5-carbaldehyde (11)

### 1-(3-Dimethylamino-propyl)-1-(4-fluoro-phenyl)-1,3-dihydro-isobenzofuran-5-carbaldehyde (8a, 11)

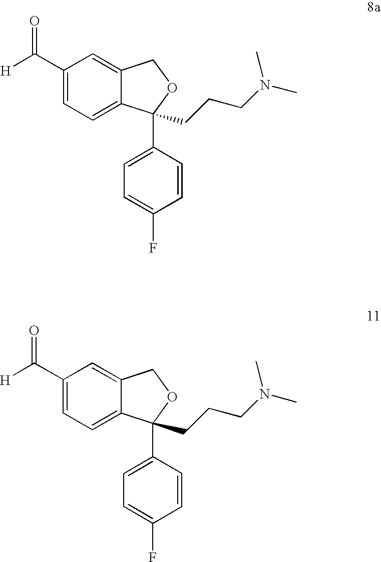

In a round-bottomed flask equipped with magnetic bar and condenser, acetic acid (20 mL) and aqueous hydrobromic acid (10 mL, 48% w/w) were added to a solution of S-isobenzfuran derivatives (7) (3.7 g, 9.5 mmol) in toluene (15 mL) (5 mmol of substrate, 10 mL of acetic acid, 5 mL of hydrobromic acid 48% w/w). The two-phase mixture was stirred overnight at room temperature. The mixture was cautiously poured into an aqueous NaOH-ice mixture. The basified aqueous solution was then extracted with ethyl acetate (3×100 mL) and the collected organic layers were washed with water (3×40 mL), brine (2×40 mL) and then dried (MgSO₄), filtered and concentrated under reduced pressure affording S-5-aldehyde-isobenzofuran derivative (8a) as red oil (3.0 g, 97%). The oxalate salt was obtained by precipitation with oxalic acid.

The same procedure was used to synthesised R-5-aldehyde-isobenzofuran derivative (11) (yield 96%) and racemic 5-aldehyde-isobenzofuran derivative (yield 97%) from (10) and its racemate.

### Free Base:

¹H (300 MHz, CDCl₃) (δ ppm): 10.00 (s, 1H), 7.81 (d, J_{HH}=7.7 Hz), 7.73 (s, 1H), 7.49-7.43 (m, 3H), 7.00 (t, J_{HH}=8.6 Hz, J_{HF}=8.6 Hz, 2H), 5.25-5.15 (AB SYSTEM, 2H), 2.30-2.16 (m, 4H), 2.14 (s, 6H), 1.56-1.26 (m, 2H).

¹³C (75 MHz, CDCl₃) (δ ppm): 192, 162 (d, ¹J_{CF}=246 Hz), 151, 141, 140 (d, 4J_{CF}=3 Hz), 137, 130, 127 (d, ³J_{CF}=8 Hz), 123, 122, 115 (d, ²J_{CF}=21 Hz), 91, 72, 60, 46 (2C), 39, 22.

### Oxalate Salt:

¹H (500 MHz, CDCl₃) (δ ppm): 10.00 (s, 1H), 7.83 (d, J_{HH}=8.0 Hz), 7.73 (s, 1H), 7.48 (d, J_{HH}=8.0 Hz), 7.44 (dd, J_{HH}=8.5 Hz, J_{HF}=5.2 Hz, 2H), 7.02 (t, J_{HH}=8.5 Hz, J_{HF}=8.5 Hz,

1. A method for the preparation of a compound of formula VI 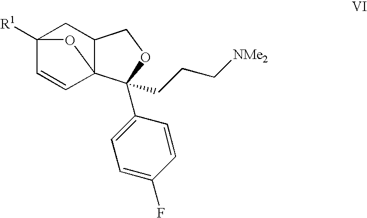
wherein R¹ is selected from functionalities that can be transformed into a nitrile group by conventional methods,
comprising allowing a compound of formula V 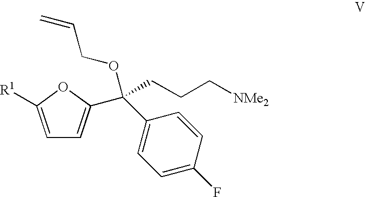
wherein R¹ is as defined above, to react to produce said compound of formula VI, optionally by heating, optionally in the presence of a Lewis acid and optionally in a suitable solvent.

2. The method according to claim 1 wherein the compound of formula VI is a compound of formula VIa or VIb 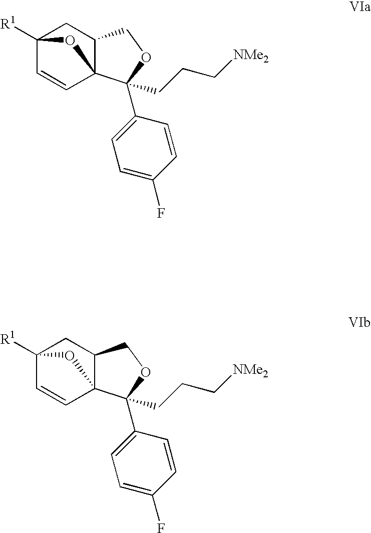
or any mixture of VIa and VIb, wherein R¹ is as defined above.

3. The method according to claim 1 wherein R¹ is selected from carboxylic acid derivatives, amides, oxazolines, carbaldehyde derivatives, and halogens.

4. The method according to claim 1 wherein R¹ is 1,3-dioxolan-2-yl.

5. The method according to claim 1 wherein the Lewis acid is selected from BF₃.Et₂O or anhydrous ZnCl₂, TiCl₄, AlCl₃, SnCl₄ or the likes.

6. The method according to claim 1 wherein the solvent is selected from CH₂Cl₂, CHCl₃ or toluene or the likes.

7. The method according to claim 1 wherein the compound of formula V is prepared by reacting a compound of formula IV 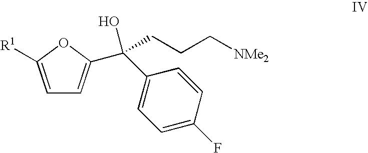
wherein R¹ is as defined above, with an allylating agent.

8. The method according to claim 7 wherein the allylating agent is selected from allyl bromide or allyl chloride.

9. The method according to claim 7 wherein the compound of formula IV is prepared by resolution of a compound of formula III 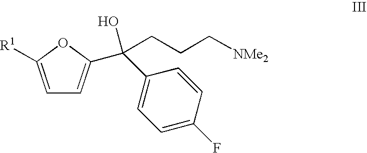
wherein R¹ is as defined above.

10. The method according to claim 9 wherein the resolution is selected from classic resolution, enzymatic resolution or chiral chromatography, such as simulated moving bed resolution.

11. The method according to claim 9 wherein the compound of formula III is prepared by reacting a compound of formula II 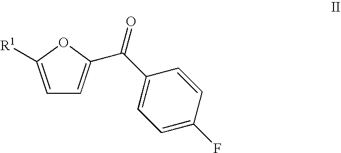
wherein R¹ is as defined above, with dimethylaminopropyl magnesium chloride.

12. The method according to claim 11 wherein the compound of formula II is prepared by reacting a compound of formula I 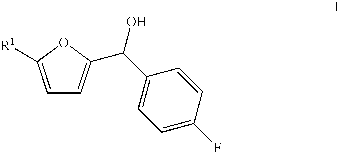
wherein R¹ is as defined above, with an oxidising agent in a suitable solvent.

13. The method according to claim 12 wherein the oxidising agent is manganese dioxide.

14. The method according to claim 12 wherein the solvent is dichloromethane.

15. The method according to claim 12 wherein the compound of formula I is prepared by reacting a compound of formula IX 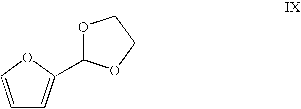
and a compound of formula X 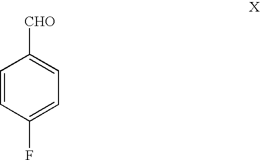
in the presence of a strong base in a suitable solvent.

16. The method according to claim 15 wherein the strong base is an organometallic agent.

17. The method according to claim 15 wherein the strong base is selected from LDA, LHMDS, methyl lithium, butyl lithium, n-butyl lithium, n-hexyl lithium or cyclohexyl lithium.

18. The method according to claim 15 wherein the solvent is THF.

19. The method according to claim 1 wherein the compound of formula VI is reacted under acidic conditions to produce a compound of formula VII 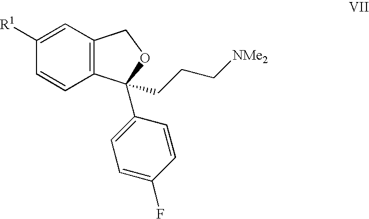
wherein R¹ is as defined above.

20. The method according to claim 19 wherein the acidic conditions are generated by an acid selected from Lewis acids, organic acids or mineral acids or a mixture thereof.

21. The method according to claim 19 wherein R¹ of the compound of formula VII is transformed into a nitrile group to produce escitalopram, a compound of formula VIII

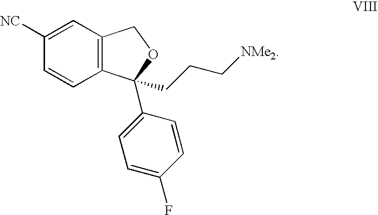

22. The method of claim 21 wherein the compound of formula VIII is optionally further purified and optionally converted to a pharmaceutically acceptable form.

23. A method for the manufacturing of escitalopram comprising one or more of the methods according to claim 1.

24. A compound of formula VI 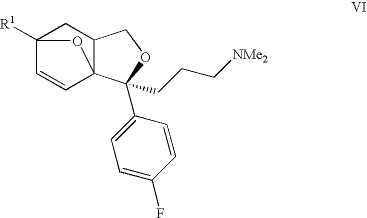
wherein R¹ is selected from functionalities that can be transformed into a nitrile group by conventional methods.

25. A compound according to claim 24 wherein the compound is S-{3-[7-[1,3]dioxolan-2-yl-2-(4-fluoro-phenyl)-3,10-dioxa-tricyclo[5.2.1.0^{1,5}]dec-8-en-2-yl]-propyl}-dimethyl-amine.

26. A compound of formula V 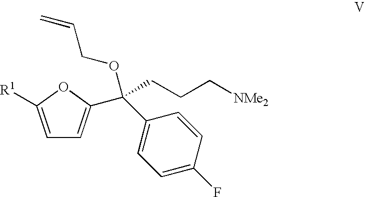
wherein R¹ is selected from functionalities that can be transformed into a nitrile group by conventional methods as defined above.

27. A compound according to claim 26 wherein the compound is S-[4-allyloxy-4-(5-[1,3]dioxolan-2-yl-furan-2-yl)-4-(4-fluoro-phenyl)-butyl]-dimethyl-amine.

28. A compound of formula IV 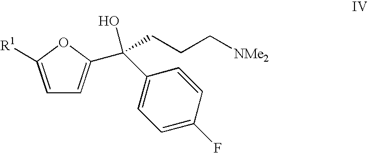
wherein R¹ is selected from functionalities that can be transformed into a nitrile group by conventional methods as defined above.

29. A compound according to claim 28 wherein the compound is S-4-dimethylamino-1-(5-[1,3]dioxolan-2-yl-furan-2-yl)-1-(4-fluoro-phenyl)-butan-1-ol.

30. A compound of formula III 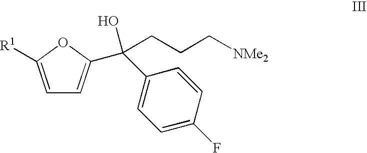
wherein R¹ is selected from functionalities that can be transformed into a nitrile group by conventional methods as defined above.

31. A compound according to claim 30 wherein the compound is 4-dimethylamino-1-(5-[1,3]dioxolan-2-yl-furan-2-yl)-1-(4-fluoro-phenyl)-butan-1-ol.

32. A compound of formula II 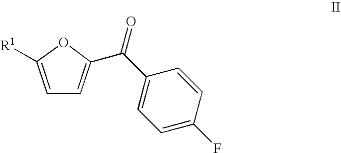
wherein R¹ is selected from functionalities that can be transformed into a nitrile group by conventional methods as defined above.

33. A compound according to claim 32 wherein the compound is (5-[1,3]dioxolan-2-yl-furan-2-yl)-(4-fluoro-phenyl)-methanone.

34. A compound of formula I 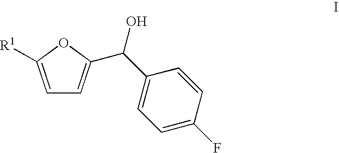
wherein R¹ is selected from functionalities that can be transformed into a nitrile group by conventional methods.

35. A compound according to claim 34 wherein the compound is (5-[1,3]dioxolan-2-yl-furan-2-yl)-(4-fluoro-phenyl)-methanol.

36. (canceled)

37. A pharmaceutical composition comprising escitalopram produced by a the methods according to claim 1.

38. The method according to claim 3, wherein R¹ is selected from —COOR² (R² is selected from C₁₋₆ alkyl, optionally substituted aryl and optionally substituted heteroaryl), CONHR³ (R³ is selected from hydrogen and C₁₋₆ alkyl), —CHO, dioxolans, acetals, aminals, Cl, Br, and I.
